(19) United States
(12) Patent Application Publication
(10) Pub. No.: US 2020/0305819 A1
(43) Pub. Date: Oct. 1, 2020

(54) Radiation Image Processing Apparatus and Radiation Image Processing Method

(71) Applicant: Shimadzu Corporation, Kyoto (JP)

(72) Inventors: Takanori YOSHIDA, Kyoto (JP); Keiichi GOTO, Kyoto (JP)

(21) Appl. No.: 16/366,803

(22) Filed: Mar. 27, 2019

(51) Int. Cl. A61B 6/00; A61B 6/03; A61F 2/82

(52) U.S. Cl. CPC A61B 6/487; A61B 6/5205; A61F 2/82; A61B 6/032; A61B 6/504

(57) ABSTRACT

A radiation image processing apparatus includes an image processing unit. The image processing unit is configured to superimpose a similar fluoroscopic image on a reference fluoroscopic image so as to create a device emphasis image in which a stent introduced into a subject is imaged in an emphasized state.

### CROSS-REFERENCE TO RELATED APPLICATIONS

The related application number JP2016-182752, entitled “Radiation image processing apparatus and radiation image processing method”, filed on Sep. 20, 2016, invented by Takanori Yoshida, and Keiichi Goto, upon which this patent application is based is hereby incorporated by reference.

### BACKGROUND OF THE INVENTION

### Field of the Invention

The present invention relates to a radiation image processing apparatus and a radiation image processing method.

### Background Art

In the related art, there is a radiation image processing apparatus which processes a fluoroscopic image obtained by imaging a medical device introduced into a subject. Such a radiation image processing apparatus is disclosed in, for example, JP-T-2005-510288.

JP-T-2005-510288 discloses an image processing apparatus which processes an X-ray image (fluoroscopic image) of a balloon and a guide wire (medical devices) introduced into the body of a subject (patient) in intervention treatment. In this image processing apparatus, a guide wire tip end or a marker (feature point) such as a balloon marker is extracted from the current image, and the current image is positioned with a reference image on the basis of a marker position. In the image processing apparatus, a plurality of positioned images are temporally integrated to be superimposed on each other.

However, the image processing apparatus disclosed in JP-T-2005-510288 has a problem that images are temporally integrated even in a case where a medical device is repeatedly deformed due to disturbance factors such as a heartbeat and breathing of a subject. In this case, since images of the medical device repeatedly deformed are superimposed on each other, and thus an image in which the medical device is blurred is created, there may be a problem in that the medical device cannot be sufficiently emphasized and displayed.

The present invention has been made in order to solve the problem, and an object of the present invention is to provide a radiation image processing apparatus and a radiation image processing method capable of acquiring a device emphasis image in which a medical device is sufficiently emphasized regardless of a disturbance factor.

### SUMMARY OF THE INVENTION

In order to achieve the object, according to a first aspect of the present invention, there is provided a radiation image processing apparatus including an image generation unit that consecutively generates fluoroscopic images based on a detection signal for radiation which is transmitted through a subject; and an image processing unit that performs image processing on the fluoroscopic images which are consecutively generated by the image generation unit, in which the image processing unit is configured to select a reference fluoroscopic image used as a reference from among the plurality of consecutively generated fluoroscopic images, analyze the plurality of consecutively generated fluoroscopic images so as to select a similar fluoroscopic image similar to the reference fluoroscopic image from among the plurality of fluoroscopic images, and superimpose the similar fluoroscopic image on the reference fluoroscopic image so as to create a device emphasis image in which a medical device introduced into the subject is imaged in an emphasized state.

In the radiation image processing apparatus according to the first aspect of the present invention, as described above, the image processing unit is configured to select a reference fluoroscopic image used as a reference from among the plurality of consecutively generated fluoroscopic images, analyze the plurality of consecutively generated fluoroscopic images so as to select a similar fluoroscopic image similar to the reference fluoroscopic image from among the plurality of fluoroscopic images, and superimpose the similar fluoroscopic image on the reference fluoroscopic image so as to create a device emphasis image in which a medical device introduced into the subject is imaged in an emphasized state. Consequently, for example, even in a case where the medical device is repeatedly deformed due to a disturbance factor, the fluoroscopic image (similar fluoroscopic image) in which the medical device having a shape similar to a shape of the medical device in the reference fluoroscopic image is imaged can be analyzed (determined) to be similar to the reference fluoroscopic image by the image processing unit. On the other hand, the fluoroscopic image (dissimilar fluoroscopic image) in which the medical device having a shape which is different from a shape of the medical device in the reference fluoroscopic image is imaged can be analyzed (determined) to be dissimilar to the reference fluoroscopic image by the image processing unit. As a result, even in a case where there is a disturbance factor, since the image processing unit does not superimpose the dissimilar fluoroscopic image on the reference fluoroscopic image, and superimposes the similar fluoroscopic image on the reference fluoroscopic image, the device emphasis image in which the medical device is sufficiently emphasized can be acquired regardless of the disturbance factor.

In the radiation image processing apparatus according to the first aspect, preferably, the image processing unit is configured to select the similar fluoroscopic image similar to the reference fluoroscopic image by analyzing the plurality of fluoroscopic images on the basis of feature points of the medical device. With this configuration, the image processing unit can easily acquire the presence or absence of the similarity between the reference fluoroscopic image and the fluoroscopic image by analyzing a plurality of fluoroscopic images on the basis of the feature points of the medical device which is clearly reflected in the fluoroscopic image.

In the configuration of analyzing the fluoroscopic image on the basis of the feature points of the medical device, preferably, the image processing unit is configured to select, as the similar fluoroscopic image, the fluoroscopic image in which the similarity of a partial image including the feature points of the medical device and the vicinity of the feature points in the fluoroscopic image with respect to a reference partial image including the feature points of the medical device and the vicinity of the feature points in the reference fluoroscopic image is equal to or more than a similarity threshold value. With this configuration, since the similarity between the reference fluoroscopic image and a plurality of fluoroscopic images is determined with respect to the feature points of the medical device and the vicinity thereof in the fluoroscopic image, the fluoroscopic image in which the medical device having a shape similar to a shape of the medical device in the reference fluoroscopic image is imaged can be more reliably selected as the similar fluoroscopic image. The image processing unit selects the fluoroscopic image in which the similarity of the partial image including the feature points of the medical device and the vicinity thereof is equal to or more than a similarity threshold value, as the similar fluoroscopic image among the fluoroscopic images, and thus it is possible to easily discriminate the similar fluoroscopic image from the dissimilar fluoroscopic image other than the similar fluoroscopic image.

In the configuration of analyzing the fluoroscopic image on the basis of the feature points of the medical device, preferably, the image processing unit is configured to select, as the similar fluoroscopic image, the fluoroscopic image in which a movement amount of the feature points of the medical device in the fluoroscopic image with respect to the feature points of the medical device in the reference fluoroscopic image is equal to or less than a movement amount threshold value. With this configuration, it is possible to easily discriminate the similar fluoroscopic image from the dissimilar fluoroscopic image other than the similar fluoroscopic image on the basis of the movement amount.

In the radiation image processing apparatus according to the first aspect, preferably, the image processing unit is configured to acquire phase information of the fluoroscopic image from the fluoroscopic image, and select, as the similar fluoroscopic image, the fluoroscopic image acquired in a phase substantially matching a phase of the reference fluoroscopic image from among the plurality of fluoroscopic images on the basis of the phase information. With this configuration, the image processing unit can easily discriminate the similar fluoroscopic image from the dissimilar fluoroscopic image other than the similar fluoroscopic image without depending on feature points of the medical device. Consequently, even in a case where feature points are hardly visually recognized, it is possible to reliably discriminate the similar fluoroscopic image from the dissimilar fluoroscopic image other than the similar fluoroscopic image. It is also possible to cope with a phase deviation based on factors other than a heartbeat, such as breathing, unlike a case where matching or mismatching of a phase is determined on the basis of an electrocardiographic waveform acquired from an electrocardiogram. Consequently, it is possible to more reliably discriminate the similar fluoroscopic image from the dissimilar fluoroscopic image other than the similar fluoroscopic image.

In the radiation image processing apparatus according to the first aspect, preferably, the image processing unit is configured to consecutively update the reference fluoroscopic image so as to consecutively create different device emphasis images. With this configuration, since the device emphasis images can be output from the radiation image processing apparatus as moving images, a user (medical practitioner) using the radiation image processing apparatus can reliably visually recognize the medical device which is deformed and moved.

The radiation image processing apparatus according to the first aspect preferably further includes a storage section that stores the fluoroscopic images which are consecutively generated by the image generation unit, and, the image processing unit is preferably configured to select the similar fluoroscopic image from among the fluoroscopic images which are stored in the storage section and are generated earlier than the reference fluoroscopic image. With this configuration, the latest fluoroscopic image is used as the reference fluoroscopic image, and thus the device emphasis image in which the medical device based on the latest fluoroscopic image is sufficiently emphasized can be acquired. Consequently, a user can clearly visually recognize the medical device in the latest state.

In the radiation image processing apparatus according to the first aspect, preferably, the medical device includes a stent for blood vessel treatment, and the fluoroscopic image is a radiation image of a part which is periodically moved. In a case where body tissue including a blood vessel is moved periodically due to a heartbeat and breathing, such as intervention treatment, it is hard to sufficiently improve visibility of a stent. Thus, the present invention capable of acquiring the device emphasis image in which the stent is sufficiently emphasized is considerably useful.

According to a second aspect of the present invention, there is provided a radiation image processing method including consecutively acquiring fluoroscopic images of a subject through radiation fluoroscopic imaging; selecting a reference fluoroscopic image used as a reference from the plurality of consecutively generated fluoroscopic images; analyzing the plurality of consecutively generated fluoroscopic images so as to select a similar fluoroscopic image similar to the reference fluoroscopic image from among the plurality of fluoroscopic images; and superimposing the similar fluoroscopic image on the reference fluoroscopic image so as to create a device emphasis image in which a medical device introduced into the subject is imaged in an emphasized state.

The radiation image processing method according to the second aspect of the present invention includes selecting a reference fluoroscopic image used as a reference from the plurality of consecutively generated fluoroscopic images; analyzing the plurality of consecutively generated fluoroscopic images so as to select a similar fluoroscopic image similar to the reference fluoroscopic image from among the plurality of fluoroscopic images; and superimposing the similar fluoroscopic image on the reference fluoroscopic image so as to create a device emphasis image in which a medical device introduced into the subject is imaged in an emphasized state. Consequently, it is possible to acquire the device emphasis image in which the medical device is sufficiently emphasized regardless of a disturbance factor in the same manner as in the radiation image processing apparatus according to the first aspect.

### BRIEF DESCRIPTION OF THE DRAWINGS

FIG. 1 is a block diagram illustrating the overall configuration of a radiation imaging apparatus including an image processing apparatus according to first to third embodiments of the present invention.

FIG. 2 is a diagram illustrating a medical device including a stent.

FIG. 3 is a diagram for explaining image processing performed by the image processing apparatus.

FIG. 4 is a flowchart for explaining a flow of the image processing performed by the image processing apparatus.

FIG. 5 is a diagram for explaining image processing performed by an image processing apparatus according to the second embodiment.

FIG. 6 is a flowchart for explaining a flow of the image processing performed by the image processing apparatus.

FIG. 7 is a diagram for explaining image processing performed by an image processing apparatus according to the third embodiment.

FIG. 8 is a flowchart for explaining a flow of the image processing performed by the image processing apparatus.

### DETAILED DESCRIPTION OF THE INVENTION

Hereinafter, embodiments of the present invention will be described with reference to the drawings.

### First Embodiment

Configuration of radiation image processing apparatus

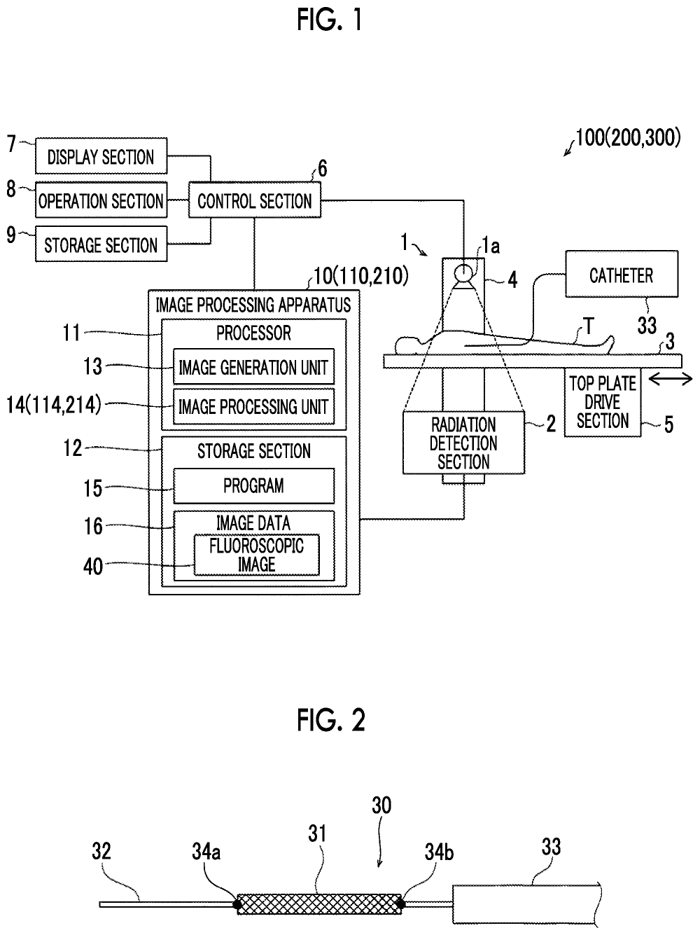

With reference to FIGS. 1 to 3, a description will be made of a configuration of an image processing apparatus 10 according to a first embodiment of the present invention. The image processing apparatus 10 is an example of a “radiation image processing apparatus” in the claims.

The image processing apparatus 10 according to the first embodiment is configured to perform image processing in real time during capturing of a fluoroscopic image in combination with a radiation imaging apparatus 100 which captures a radiation image. The radiation imaging apparatus 100 is an apparatus which applies radiation from the outside of a subject T such as a human body, and thus captures (performs radiation fluoroscopic imaging) a radiation image (fluoroscopic image) obtained by imaging the inside of the subject T. The radiation imaging apparatus 100 is an X-ray imaging apparatus which captures an X-ray image by using an X-ray which is an example of radiation.

The radiation imaging apparatus 100 includes an irradiation section 1 which irradiates the subject T with radiation (X-ray) and a radiation detection section 2 which detects the radiation transmitted through the subject T. The irradiation section 1 and the radiation detection section 2 are disposed to be opposed to each other with a top plate 3, interposed therebetween, on which the subject T is mounted. The irradiation section 1 and the radiation detection section 2 are movably supported by a movement mechanism 4. The top plate 3 is movable in a horizontal direction by a top plate drive section 5. The irradiation section 1, the radiation detection section 2, and the top plate 3 are moved via the movement mechanism 4 and the top plate drive section 5, and thus an imaging region is moved. The imaging region is an imaging target region in the subject T in order to perform an examination or treatment. The radiation imaging apparatus 100 includes a control section 6 which controls the movement mechanism 4 and the top plate drive section 5.

The irradiation section 1 includes a radiation source 1a. The radiation source 1a is an X-ray tube which is connected to a high voltage generation section (not illustrated), and generates an X-ray as a result of a high voltage being applied thereto. The radiation source 1a is disposed in a state in which an X-ray emission direction is directed to a detection surface of the radiation detection section 2. The irradiation section 1 is connected to the control section 6. The control section 6 controls the irradiation section 1 according to preset imaging conditions such as a tube voltage, a tube current, and a time interval of X-ray irradiation, so as to generate an X-ray from the radiation source 1a.

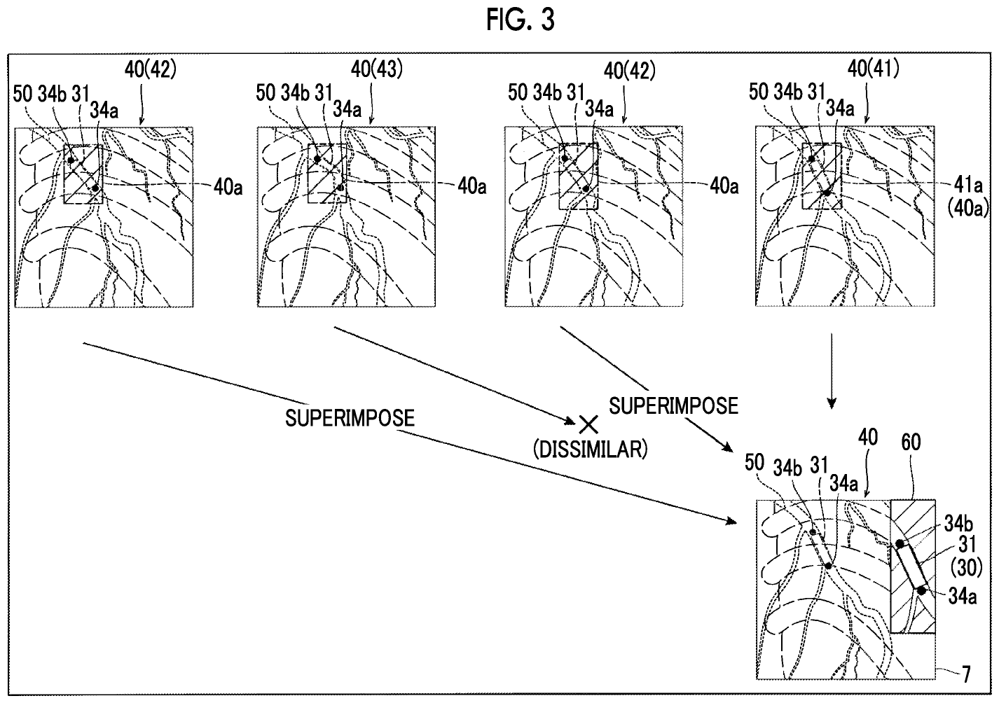

The radiation detection section 2 detects an X-ray which is applied from the irradiation section 1 and is transmitted through the subject T, and outputs a detection signal corresponding to a detected X-ray intensity. The radiation detection section 2 is configured with, for example, a flat panel detector (FPD). The radiation detection section 2 outputs an X-ray detection signal with a predetermined resolution to the image processing apparatus 10. The image processing apparatus 10 acquires the X-ray detection signal from the radiation detection section 2, and generates a fluoroscopic image 40 (refer to FIG. 3).

The control section 6 is a computer configured to include a central processing unit (CPU), a read only memory (ROM), and a random access memory (RAM). The control section 6 controls each section of the radiation imaging apparatus 100 by the CPU executing a predetermined control program. The control section 6 performs control of the irradiation section 1 and the image processing apparatus 10 or drive control of the movement mechanism 4 and the top plate drive section 5.

The radiation imaging apparatus 100 includes a display section 7, an operation section 8, and a storage section 9. The display section 7 is a monitor such as a liquid crystal display. The operation section 8 is configured to include, for example, a keyboard, a mouse, and a touch panel, or other controllers. The storage section 9 is configured with a storage device such as a hard disk drive. The control section 6 is configured to perform control of displaying an image generated by the image processing apparatus 10 on the display section 7. The control section 6 is configured to receive an input operation using the operation section 8. The storage section 9 is configured to store image data, imaging conditions, and various set values. Each of the display section 7 and the operation section 8 may be provided in the image processing apparatus 10.

The image processing apparatus 10 is a computer configured to include a processor 11 such as a CPU or a graphics processing unit (GPU), and a storage section 12 such as a ROM and a RAM. In other words, the image processing apparatus 10 is configured to cause the processor 11 to execute an image processing program stored in the storage section 12. The image processing apparatus 10 may be integrally configured with the control section 6 by causing the same hardware (CPU) as the control section 6 to execute the image processing program.

The storage section 12 stores a program 15 (image processing program) for causing a computer to function as the image processing apparatus 10. In the first embodiment, the storage section 12 is configured to store image data 16 including fluoroscopic images 40 which are consecutively generated by an image generation unit 13 which will be described later.

The image processing apparatus 10 includes the image generation unit 13 and an image processing unit 14 as functions realized by executing the program 15. The image generation unit 13 and the image processing unit 14 may be configured separately from each other by dedicated processors.

The image generation unit 13 is configured to generate the fluoroscopic image 40 based on a detection signal of radiation transmitted through the subject T. The image generation unit 13 consecutively generates the fluoroscopic images 40 in a moving image form on the basis of detection signals from the radiation detection section 2. In other words, X-rays are intermittently applied to the subject T from the irradiation section 1 at a predetermined time interval, and X-rays transmitted through the subject T are sequentially detected by the radiation detection section 2. The image generation unit 13 images detection signals which are sequentially output from the radiation detection section 2, and thus consecutively generates the fluoroscopic images 40 at a predetermined frame rate. The frame rate is, for example, about 15 FPS to 30 FPS.

The image processing unit 14 is configured to perform image processing on the fluoroscopic image 40 generated by the image generation unit 13. Details of the image processing will be described later.

In the first embodiment, the image processing apparatus 10 (radiation imaging apparatus 100) is configured to generate the fluoroscopic image 40 of a medical device 30 (refer to FIG. 2) introduced into the subject T. In the first embodiment, the fluoroscopic image 40 is a radiation image of a part which is periodically moved due to a heartbeat and breathing of the subject.

As illustrated in FIG. 2, the medical device 30 introduced into the subject T includes a stent 31 for blood vessel treatment. The stent 31 is used for, for example, coronary artery (cardiovascular) intervention treatment. In the coronary artery intervention treatment, treatment is performed by inserting a catheter 33 having a guide wire 32 therein into a blood vessel of the subject T, and causing the catheter 33 to reach a coronary artery of the heart via the blood vessel.

The stent 31 has a tubular shape having a mesh structure made of thin metal or resin. The stent 31 is disposed in a stenosed part of a blood vessel, and is placed in the blood vessel as a result of being expanded by using a balloon from the inside, so as to support the stenosed blood vessel from the inside. The stent 31 made of resin has a small difference in X-ray transmission with respect to peripheral body tissue and blood vessel. Consequently, the stent 31 made of resin has lower visibility in the fluoroscopic image 40 than the stent 31 made of metal.

Therefore, the stent 31 having a mesh structure is hardly to be reflected in the fluoroscopic image 40, and thus a pair of markers 34a and 34b of which radiation transmission is low (or radiation transmission is zero) is provided at the stent 31 as marks. The marker 34a is provided at one end part of the stent 31 in a longitudinal direction. The marker 34b is provided at the other end part of the stent 31 in the longitudinal direction. The markers 34a and 34b are examples of “feature points” in the claims.

In the coronary artery intervention treatment, a doctor (medical practitioner) sends the catheter 33 to a coronary artery of the heart while referring to the fluoroscopic images 40 which are moving images generated in real time by the image processing apparatus 10 (radiation imaging apparatus 100). During treatment, it is necessary to specify a stenosed part, to determine positions of the stent 31 and the balloon for blood vessel expansion in the stenosed part, and to check the stent 31 after being placed. Since blood in a blood vessel and peripheral body tissue have a small difference in X-ray transmission, and thus a blood vessel portion has low visibility in the fluoroscopic image 40.

Here, as illustrated in FIG. 3, positions of the markers 34a and 34b reflected as black dots in the typical fluoroscopic image 40 can be recognized. However, in the typical fluoroscopic image 40, it is not easy to clearly recognize a position of the actual stent 31, and details of a peripheral region such as a blood vessel 50 and body tissue in the vicinity of the markers 34a and 34b. The actual stent 31 has low visibility, and is also deformed to be bent in a blood vessel due to disturbance factors such as a heartbeat and breathing or is moved in accordance with movement of the blood vessel. As a result, the fluoroscopic image 40 in which a shape of the stent 31 and a peripheral region are clearly imaged cannot be obtained.

Therefore, in the first embodiment, the image processing unit 14 performs image combination (superimposition) on a plurality of fluoroscopic images 40 similar to each other among the fluoroscopic images 40. Consequently, the image processing unit 14 is configured to create a device emphasis image 60 in which the stent 31 is emphasized.

Image Processing on Fluoroscopic Image

Specifically, the image processing unit 14 is configured to perform a process of selecting a reference fluoroscopic image 41 from among a plurality of fluoroscopic images 40 which are consecutively generated, a process of analyzing the plurality of fluoroscopic images 40 which are consecutively generated and selecting a similar fluoroscopic image 42 similar to the reference fluoroscopic image 41 from among the plurality of fluoroscopic images 40 which are consecutively generated, and a process of creating the device emphasis image 60 by superimposing the similar fluoroscopic image 42 on the reference fluoroscopic image 41. Hereinafter, each process will be described in detail.

Description of Reference Fluoroscopic Image

The reference fluoroscopic image 41 is the latest fluoroscopic image 40 which is newly captured in a plurality of fluoroscopic images 40 which are consecutively generated. The reference fluoroscopic image is not limited to the latest fluoroscopic image 40. For example, the image processing unit 14 may select the fluoroscopic image 40 which is generated at a predetermined timing from among a plurality of fluoroscopic images 40 which are consecutively generated, as the reference fluoroscopic image 41.

The image processing unit 14 acquires position coordinates of the markers 34a and 34b in the reference fluoroscopic image 41. The markers 34a and 34b may be detected by using a well-known image recognition technique. The image processing unit 14 acquires a reference partial image 41a including the markers 34a and 34b and the vicinity thereof on the basis of the position coordinates of the markers 34a and 34b. The reference partial image 41a includes a line segment connecting the markers 34a and 34b to each other. Consequently, the reference partial image 41a includes the entire stent 31.

Description of Similar Fluoroscopic Image

The similar fluoroscopic image 42 is the fluoroscopic image 40 which is similar to the reference fluoroscopic image 41 and is selected from among a plurality of fluoroscopic images 40 which are consecutively generated. The similar fluoroscopic image 42 is one of the fluoroscopic images 40 which are acquired earlier than the reference fluoroscopic image 41, and is selected from among a predetermined frame number (plurality of) fluoroscopic images 40 of the fluoroscopic images 40 stored in the storage section 12.

Here, in the first embodiment, the image processing unit 14 is configured to analyze a predetermined frame number of fluoroscopic images 40 which are consecutively generated, and thus to select the similar fluoroscopic image 42 similar to the reference fluoroscopic image 41 from among a plurality of fluoroscopic images 40. Specifically, the image processing unit 14 acquires position coordinates of the markers 34a and 34b in any fluoroscopic image 40. The image processing unit 14 acquires a partial image 40a including the markers 34a and 34b and the vicinity thereof on the basis of the position coordinates of the markers 34a and 34b. The partial image 40a includes a line segment connecting the markers 34a and 34b to each other. Consequently, the partial image 40a includes the entire stent 31.

Thereafter, the image processing unit 14 analyzes the partial image 40a, so as to acquire the similarity of the partial image 40a for the reference partial image 41a, and selects the fluoroscopic image 40 in which the similarity of the partial image 40a is equal to or more than a similarity threshold value, as the similar fluoroscopic image 42. The similarity of the partial image 40a for the reference partial image 41a may be acquired by using general pattern matching such as normalized cross-correlation (NCC). The similarity threshold value is set for each pattern matching.

The image processing unit 14 may be configured to determine the resemblance of the fluoroscopic image 40 for the entire reference fluoroscopic image 41 instead of determining the resemblance of the partial image 40a including only the markers 34a and 34b and the vicinity thereof.

The image processing unit 14 performs determination of the resemblance based on the similarity on each of a predetermined frame number of fluoroscopic images 40, and selects the similar fluoroscopic image 42 from among predetermined frame number of fluoroscopic images 40.

Finally, the image processing unit 14 superimposes the selected similar fluoroscopic image 42 on the reference fluoroscopic image 41, and does not superimpose a non-selected fluoroscopic image 40 (dissimilar fluoroscopic image 43) thereon. Consequently, the image processing unit 14 creates the device emphasis image 60 in which the stent 31 introduced into the subject T is imaged in an emphasized state. In the device emphasis image 60, even a fine structure of the stent 31 having a low visibility is clearly displayed, and body tissue such as a peripheral blood vessel of the stent 31 is also clearly displayed.

Positions of the markers 34a and 34b are not substantially changed between the similar fluoroscopic images 40. Consequently, the device emphasis image 60 in which the stent 31 is emphasized can be acquired even if positioning of respectively aligning positions of the markers 34a and 34b in the superimposed similar fluoroscopic image 42 with positions of the markers 34a and 34b in the reference fluoroscopic image 41.

The device emphasis image 60 is output to the display section 7 along with the latest fluoroscopic image (reference fluoroscopic image 41) under the control of the control section 6. Consequently, the latest fluoroscopic image 40 is displayed on the display section 7, and the device emphasis image 60 is displayed on the display section 7 in a display region which is different from that of the latest fluoroscopic image 40. The device emphasis image 60 may be an image having the same range as that of the partial image 40a, and may be an image having a range which is different from that of the partial image 40a as long as the entire stent 31 is included therein.

The image processing unit 14 updates the new fluoroscopic image 40 to the reference fluoroscopic image 41, creates the new device emphasis image 60 by performing the same processes as those described above, and outputs the new device emphasis image 60 to the display section 7. Consequently, different device emphasis images 60 are consecutively created by the image processing unit 14, and are displayed on the display section 7 as moving images.

Process Operation of Image Processing Apparatus

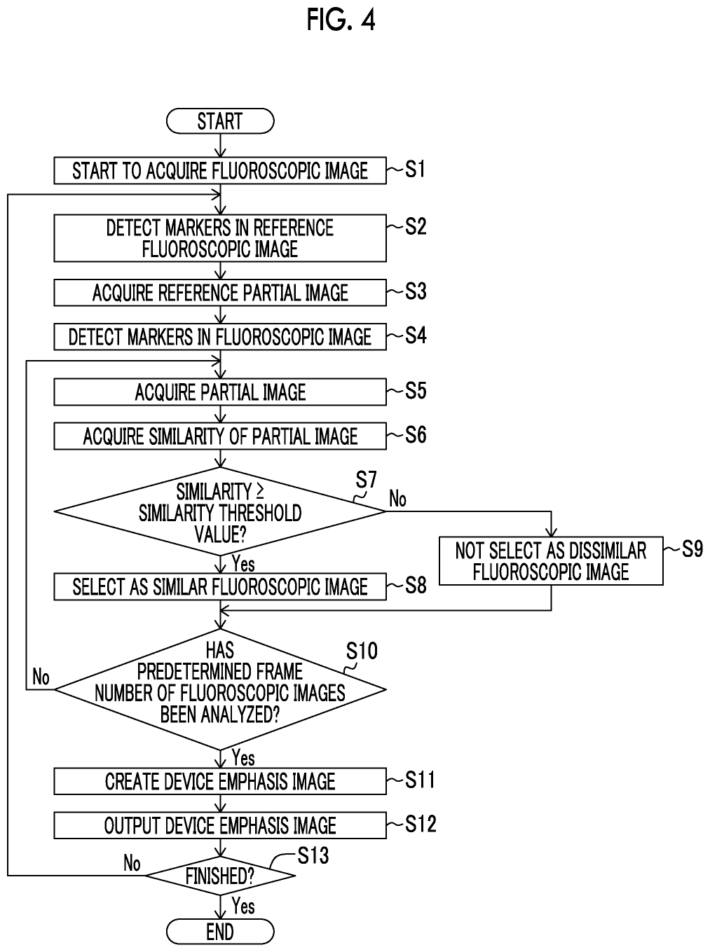

Next, with reference to FIG. 4, a description will be made of a process operation of the image processing apparatus 10 according to the first embodiment.

In step S1 in FIG. 4, the image processing apparatus 10 starts to acquire the fluoroscopic image 40. In other words, a detection signal is acquired from the radiation detection section 2 which detects an X-ray which is applied from the irradiation section 1 and is transmitted through the subject T. The image generation unit 13 generates the fluoroscopic image 40 on the basis of the acquired detection signal. The fluoroscopic image 40 is consecutively generated in the frame unit as a moving image, and is sequentially output to the image processing unit 14 and is also stored in the storage section 12.

In step S2, the image processing unit 14 selects the generated new fluoroscopic image 40 as the reference fluoroscopic image 41. The image processing unit 14 detects the markers 34a and 34b in the reference fluoroscopic image 41 through image recognition, and thus acquires position coordinates of the markers 34a and 34b in the reference fluoroscopic image 41. In step S3, the image processing unit 14 acquires the reference partial image 41a including the markers 34a and 34b and the vicinity thereof on the basis of the position coordinates of the markers 34a and 34b in the reference fluoroscopic image 41.

In step S4, the image processing unit 14 detects the markers 34a and 34b in the fluoroscopic image 40 through image recognition from a predetermined fluoroscopic image 40 stored in the storage section 12, and acquires position coordinates of the markers 34a and 34b in the reference fluoroscopic image 41. In step S5, the image processing unit 14 acquires the partial image 40a including the markers 34a and 34b and the vicinity thereof in the predetermined fluoroscopic image 40 on the basis of the position coordinates of the markers 34a and 34b in the fluoroscopic image 40. In step S6, the image processing unit 14 analyzes the partial image 40a so as to acquire the similarity of the partial image 40a for the reference partial image 41a.

In step S7, the image processing unit 14 determines whether or not the acquired similarity of the partial image 40a is equal to or more than a similarity threshold value. In a case where the similarity is equal to or more than the similarity threshold value, in step S8, the image processing unit 14 regards and selects the fluoroscopic image 40 as the similar fluoroscopic image 42, and proceeds to step S10. In a case where the similarity is less than the similarity threshold value, in step S9, the image processing unit 14 regards the fluoroscopic image 40 as the dissimilar fluoroscopic image 43 and does not select the fluoroscopic image 40, and proceeds to step S10.

In step S10, the image processing unit 14 determines whether or not the similarity is determined by analyzing a predetermined frame number of fluoroscopic images 40. In a case where it is determined that the predetermined frame number of fluoroscopic images 40 is not analyzed, the flow returns to step S5, and the similarity of another fluoroscopic image 40 is determined. In a case where it is determined that the predetermined frame number of fluoroscopic images 40 is analyzed, in step S11, the image processing unit 14 creates the device emphasis image 60 by superimposing the selected similar fluoroscopic image 42 on the reference fluoroscopic image 41 instead of superimposing the non-selected dissimilar fluoroscopic image 43 thereon.

In step S12, the image processing unit 14 outputs the created device emphasis image 60 to the display section 7 (control section 6). Consequently, the device emphasis image 60 is displayed on the display section 7.

In step S13, the image processing unit 14 determines whether or not an instruction for finishing the image processing is given from the control section 6 by a doctor (medical practitioner) or the like operating the operation section 8. In a case where the instruction for finishing the image processing is not given, the image processing unit 14 returns to step S2, updates the new fluoroscopic image 40 to the reference fluoroscopic image 41, and creates a new device emphasis image 60. Consequently, different device emphasis images 60 are consecutively created by the image processing unit 14. The device emphasis image 60 is updated. In a case where the instruction for finishing the image processing is given, the process operation of the image processing apparatus 10 is finished.

Effects of First Embodiment

In the first embodiment, the following effects can be achieved.

In the first embodiment, as mentioned above, the reference fluoroscopic image 41 which is used as a reference is selected from among a plurality of fluoroscopic images 40 which are consecutively generated. The plurality of consecutively generated fluoroscopic images 40 are analyzed, and thus the similar fluoroscopic image 42 similar to the reference fluoroscopic image 41 is selected from among the plurality of fluoroscopic images 40. The image processing unit 14 is configured to create the device emphasis image 60 in which the stent 31 introduced into the subject T is imaged in an emphasized state by superimposing the similar fluoroscopic image 42 on the reference fluoroscopic image 41. Consequently, for example, even in a case where the stent 31 is repeatedly deformed due to a disturbance factor, the fluoroscopic image 40 (similar fluoroscopic image 42) in which the stent 31 having a shape similar to a shape of the stent 31 in the reference fluoroscopic image 41 is imaged can be analyzed (determined) to be similar to the reference fluoroscopic image 41 by the image processing unit 14. On the other hand, the fluoroscopic image 40 (dissimilar fluoroscopic image 43) in which the stent 31 having a shape which is different from a shape of the stent 31 in the reference fluoroscopic image 41 is imaged can be analyzed (determined) to be dissimilar to the reference fluoroscopic image 41 by the image processing unit 14. As a result, in a case where there is a disturbance factor, since the image processing unit 14 does not superimpose the dissimilar fluoroscopic image 43 on the reference fluoroscopic image 41, and superimposes the similar fluoroscopic image 42 on the reference fluoroscopic image 41, the device emphasis image 60 in which the stent 31 is sufficiently emphasized can be acquired regardless of the disturbance factor.

In the first embodiment, as described above, the image processing unit 14 is configured to analyze a plurality of fluoroscopic images 40 on the basis of the markers 34a and 34b of the stent 31 and thus to select the similar fluoroscopic image 42 similar to the reference fluoroscopic image 41. Consequently, the image processing unit 14 can easily acquire the presence or absence of the similarity between the reference fluoroscopic image 41 and the fluoroscopic image 40 by analyzing the plurality of fluoroscopic images 40 on the basis of the markers 34a and 34b of the stent 31 which is clearly reflected in the fluoroscopic image 40.

In the first embodiment, as described above, the image processing unit 14 is configured to select the fluoroscopic image 40 in which the similarity of the partial image 40a including the markers 34a and 34b of the stent 31 and the vicinity thereof in the fluoroscopic image 40 is equal to or more than a similarity threshold value for the reference partial image 41a including the markers 34a and 34b of the stent 31 and the vicinity thereof in the reference fluoroscopic image 41, as the similar fluoroscopic image 42. Consequently, since the similarity between the reference fluoroscopic image 41 and a plurality of fluoroscopic images 40 is determined with respect to the markers 34a and 34b of the stent 31 and the vicinity thereof in the fluoroscopic image 40, the fluoroscopic image 40 in which the stent 31 having a shape similar to a shape of the stent 31 in the reference fluoroscopic image 41 is imaged can be more reliably selected as the similar fluoroscopic image 42. The image processing unit 14 selects the fluoroscopic image 40 in which the similarity of the partial image 40a including the markers 34a and 34b of the stent 31 and the vicinity thereof is equal to or more than a similarity threshold value, as the similar fluoroscopic image 42 among the fluoroscopic images 40. Consequently, it is possible to easily discriminate the similar fluoroscopic image 42 from the dissimilar fluoroscopic image 43 other than the similar fluoroscopic image 42.

In the first embodiment, as described above, the image processing unit 14 is configured to consecutively create the different device emphasis images 60 by consecutively updating the reference fluoroscopic images 41. Consequently, since the device emphasis images 60 can be output from the image processing apparatus 10 as moving images, a user (medical practitioner) using the image processing apparatus 10 can reliably visually recognize the stent 31 which is deformed and moved.

In the first embodiment, as described above, the image processing unit 14 is configured to select the similar fluoroscopic image 42 from among the fluoroscopic images 40 which are stored in the storage section 12 and are generated earlier than the reference fluoroscopic image 41. Consequently, the latest fluoroscopic image 40 is used as the reference fluoroscopic image 41, and thus the device emphasis image 60 in which the stent 31 based on the latest fluoroscopic image 40 is sufficiently emphasized can be acquired. As a result, a user can clearly visually recognize the stent 31 in the latest state.

In the first embodiment, as described above, the medical device 30 includes the stent 31 for blood vessel treatment, and the fluoroscopic image 40 is a radiation image of a part which is periodically moved. In a case where body tissue including a blood vessel is moved periodically due to a heartbeat and breathing, such as intervention treatment, it is hard to sufficiently improve visibility of the stent 31. Thus, the present invention capable of acquiring the device emphasis image 60 in which the stent 31 is sufficiently emphasized is considerably useful.

### Second Embodiment

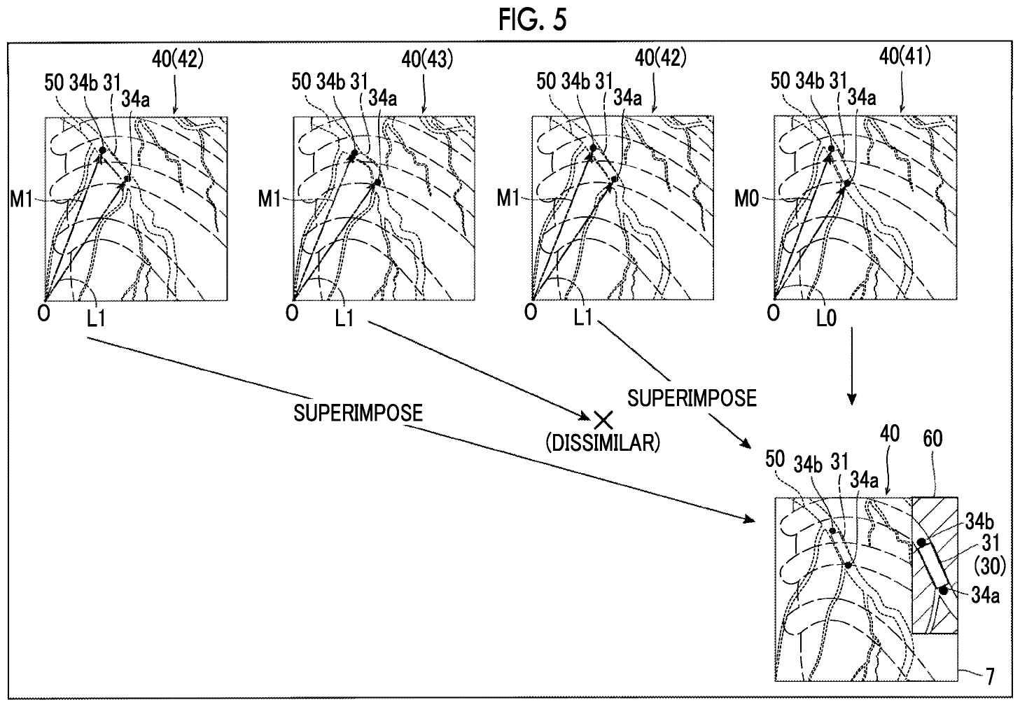

Next, with reference to FIGS. 1, 5, and 6, a second embodiment will be described. In the second embodiment, unlike the first embodiment, a description will be made of an example in which whether or not a fluoroscopic image is similar to a reference fluoroscopic image is determined on the basis of a movement amount of a marker. The same constituent element as that in the first embodiment is given the same reference numeral, and a description thereof will be omitted.

In the second embodiment, as illustrated in FIG. 1, a radiation imaging apparatus 200 includes an image processing apparatus 110 instead of the image processing apparatus 10 of the first embodiment. The image processing apparatus 110 includes an image processing unit 114 instead of the image processing unit 14 of the first embodiment as functions realized by executing the program 15. The image processing apparatus 110 is an example of a “radiation image processing apparatus” in the claims.

In the second embodiment, the image processing unit 114 is configured to determine the resemblance between the reference fluoroscopic image 41 and the fluoroscopic image 40 on the basis of a movement amount of the markers 34a and 34b.

Specifically, the image processing unit 114 acquires (analyzes) position coordinates of the markers 34a and 34b in the reference fluoroscopic image 41. The image processing unit 114 acquires respective distances L0 and M0 between an origin position O and the markers 34a and 34b on the basis of the position coordinates of the markers 34a and 34b. The origin position O is any corner of an imaging region. The corner is not moved during imaging, and the origin position O in the reference fluoroscopic image 41 substantially matches origin position O in the fluoroscopic image 40.

The image processing unit 114 acquires (analyzes) position coordinates of the markers 34a and 34b in any fluoroscopic image 40. The image processing unit 114 acquires respective distances L1 and M1 between the origin position O and the markers 34a and 34b on the basis of the position coordinates of the markers 34a and 34b.

Thereafter, the image processing unit 114 acquires a movement amount of the markers 34a and 34b of the stent 31 in the fluoroscopic image 40 for the markers 34a and 34b of the stent 31 in the reference fluoroscopic image 41. Specifically, the image processing unit 114 acquires a movement amount I in the fluoroscopic image 40 on the basis of the following Equation (1). The movement amount I may be obtained according to other methods without limitation to the following Equation (1).

I=√((L1−L0)²+(M1−M0)²) (1)

The image processing unit 114 selects the fluoroscopic image 40 in which the movement amount I in the partial image 40a is equal to or less than a movement amount threshold value, as the similar fluoroscopic image 42. The image processing unit 114 performs the determination of the resemblance based on the movement amount on each of a predetermined frame number of fluoroscopic images 40, and selects the similar fluoroscopic image 42 from among predetermined frame number of fluoroscopic images 40.

Finally, in the same manner as the image processing unit 14 of the first embodiment, the image processing unit 114 superimposes the selected similar fluoroscopic image 42 on the reference fluoroscopic image 41, and does not superimpose a non-selected fluoroscopic image 40 (dissimilar fluoroscopic image 43) thereon. Consequently, the image processing unit 114 creates the device emphasis image 60 in which the stent 31 introduced into the subject T is imaged in an emphasized state.

The rest configuration of the second embodiment is the same as that of the first embodiment.

Process Operation of Image Processing Apparatus

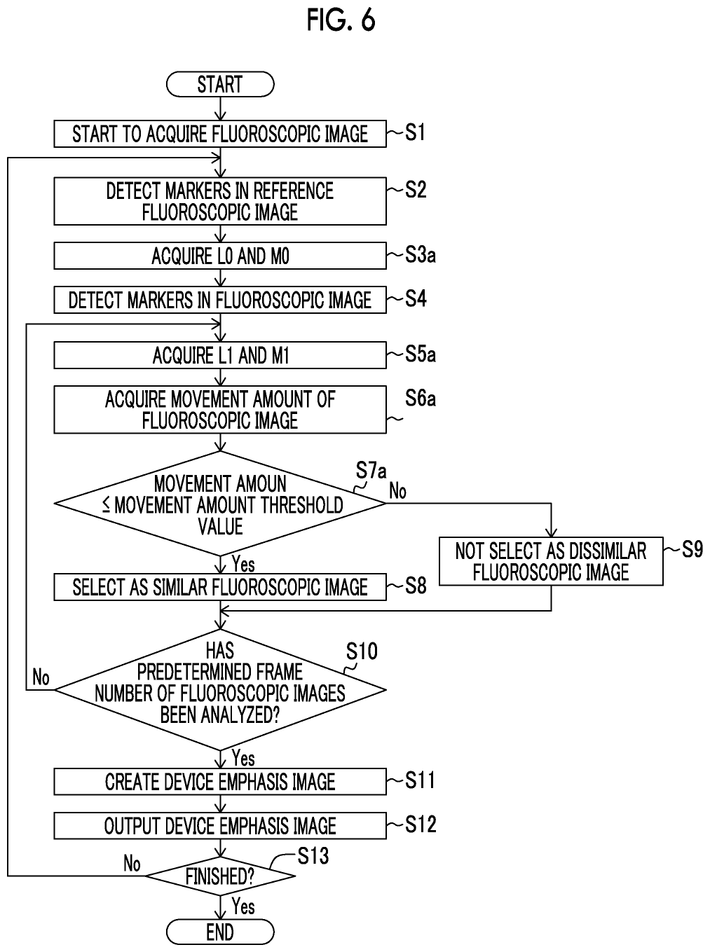

Next, with reference to FIG. 6, a description will be made of a process operation of the image processing apparatus 110 according to the second embodiment. The same process operation as that in the first embodiment is given the same reference sign (step S), and a description thereof will be omitted.

As illustrated in FIG. 6, steps S1 and S2 are the same as those in the first embodiment illustrated in FIG. 4. In step S3a, the image processing unit 114 acquires respective distances L0 and M0 between an origin position O and the markers 34a and 34b on the basis of the position coordinates of the markers 34a and 34b in the reference fluoroscopic image 41.

In step S4, the image processing unit 114 acquires position coordinates of the markers 34a and 34b in the fluoroscopic image 40. In step S5a, the image processing unit 114 acquires respective distances L1 and M1 between the origin position O and the markers 34a and 34b on the basis of the position coordinates of the markers 34a and 34b in the fluoroscopic image 40. In step S6a, the image processing unit 114 acquires the movement amount I in the fluoroscopic image 40 on the basis of the above Equation (1).

In step S7a, the image processing unit 114 determines whether or not the acquired movement amount I of the partial image 40a is equal to or less than a movement amount threshold value. In a case where the movement amount I is equal to or less than the movement amount threshold value, in step S8, the image processing unit 114 regards and selects the fluoroscopic image 40 as the similar fluoroscopic image 42, and proceeds to step S10. In a case where the movement amount I is more than the movement amount threshold value, in step S9, the image processing unit 114 regards the fluoroscopic image 40 as the dissimilar fluoroscopic image 43 and does not select the fluoroscopic image 40, and proceeds to step S10. Steps S10 to S13 are the same as those in the first embodiment illustrated in FIG. 4.

Effects of Second Embodiment

In the second embodiment, the reference fluoroscopic image 41 which is used as a reference is selected from among a plurality of fluoroscopic images 40 which are consecutively generated. The plurality of consecutively generated fluoroscopic images 40 are analyzed, and thus the similar fluoroscopic image 42 similar to the reference fluoroscopic image 41 is selected from among the plurality of fluoroscopic images 40. The image processing unit 114 is configured to create the device emphasis image 60 in which the stent 31 introduced into the subject T is imaged in an emphasized state by superimposing the similar fluoroscopic image 42 on the reference fluoroscopic image 41. Consequently, in the same manner as in the first embodiment, the device emphasis image 60 in which the stent 31 is sufficiently emphasized can be acquired regardless of the disturbance factor.

In the second embodiment, as described above, the image processing unit 114 is configured to select the fluoroscopic image 40 in which the movement amount I of the markers 34a and 34b of the stent 31 in the fluoroscopic image 40 for the markers 34a and 34b of the stent 31 in the reference fluoroscopic image 41 is equal to or less than a movement amount threshold value, as the similar fluoroscopic image 42. Consequently, it is possible to easily discriminate the similar fluoroscopic image 42 from the dissimilar fluoroscopic image 43 other than the similar fluoroscopic image 42 on the basis of the movement amount I.

### Third Embodiment

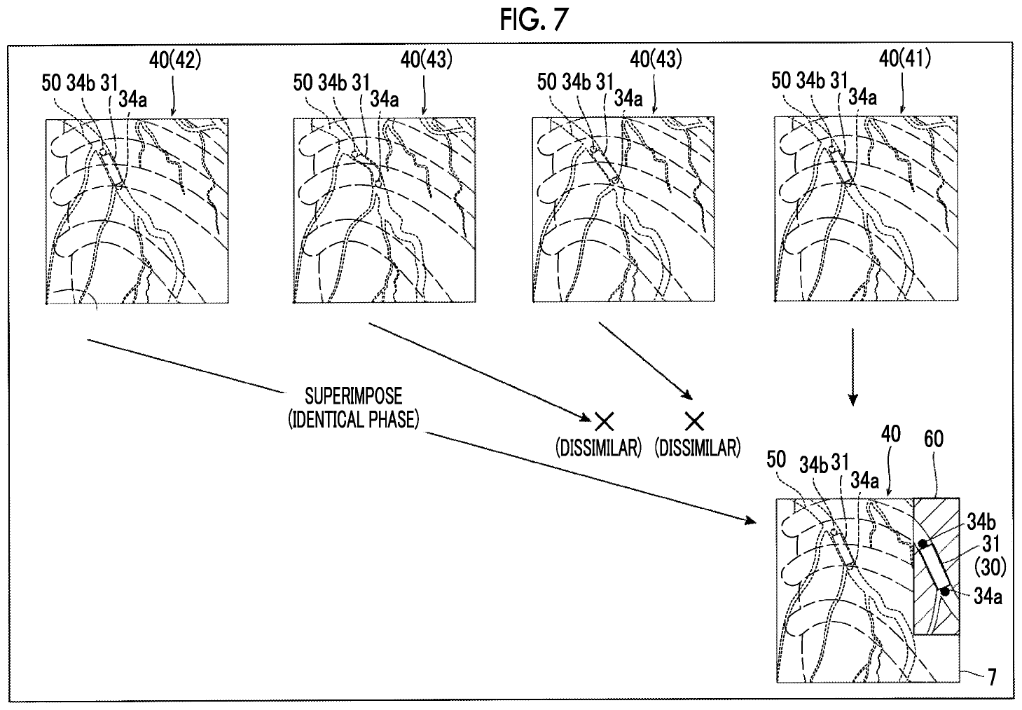

Next, with reference to FIGS. 1, 7, and 8, a third embodiment will be described. In the third embodiment, unlike the first embodiment, a description will be made of an example in which whether or not a fluoroscopic image is similar to a reference fluoroscopic image is determined on the basis of phase information of the fluoroscopic image. The same constituent element as that in the first embodiment is given the same reference numeral, and a description thereof will be omitted.

In the third embodiment, as illustrated in FIG. 1, a radiation imaging apparatus 300 includes an image processing apparatus 210 instead of the image processing apparatus 10 of the first embodiment. The image processing apparatus 210 includes an image processing unit 214 instead of the image processing unit 14 of the first embodiment as functions realized by executing the program 15. The image processing apparatus 210 is an example of a “radiation image processing apparatus” in the claims.

In the third embodiment, the image processing unit 214 is configured to determine the resemblance between the reference fluoroscopic image 41 and the fluoroscopic image 40 on the basis of phase information of the fluoroscopic image 40.

Specifically, the image processing unit 214 is configured to acquire phase information based on movement changes such as a heartbeat and breathing by analyzing each fluoroscopic image 40. The image processing unit 214 is configured to determine the resemblance between the reference fluoroscopic image 41 and the fluoroscopic image on the basis of the phase information of the fluoroscopic image 40. A method of selecting the fluoroscopic image 40 having a phase which substantially matches a phase of the reference fluoroscopic image 41 on the basis of phase information acquired from the fluoroscopic image 40 may employ the contents disclosed in detail in Japanese Patent Application No. 2015-232474 filed by the present applicant. In the present specification, the disclosure of Japanese Patent Application No. 2015-232474 is incorporated by reference.

To summarize, the image processing unit 214 extracts a plurality of (three or more) feature points reflected in the reference fluoroscopic image 41 through image recognition. Each feature point is a point which is periodically moved according to a phase. The image processing unit 214 obtains a centroid position of each feature point position, and obtains a position vector of each feature point for the centroid position. The image processing unit 214 selects the fluoroscopic image 40 having a position vector group which matches a position vector group of each feature point in the reference fluoroscopic image 41 to some extent, as the similar fluoroscopic image 42 which is generated in a substantially matching phase (identical phase). In this case, the position vector group of each feature point is phase information.

In the same manner as the image processing unit 14 of the first embodiment, the image processing unit 214 superimposes the selected similar fluoroscopic image 42 on the reference fluoroscopic image 41, and does not superimpose a non-selected fluoroscopic image 40 (dissimilar fluoroscopic image 43) thereon. Consequently, the image processing unit 214 creates the device emphasis image 60 in which the stent 31 introduced into the subject T is imaged in an emphasized state.

The rest configuration of the third embodiment is the same as that of the first embodiment.

Process Operation of Image Processing Apparatus

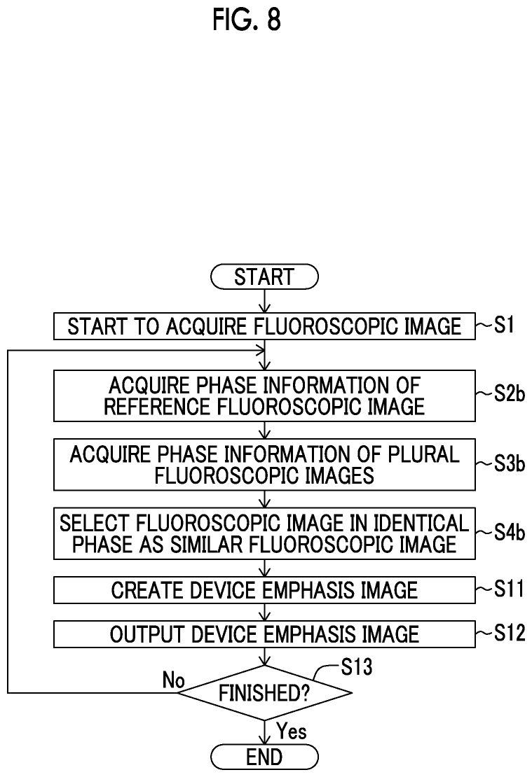

Next, with reference to FIG. 8, a description will be made of a process operation of the image processing apparatus 210 according to the third embodiment. The same process operation as that in the first embodiment is given the same reference sign (step S), and a description thereof will be omitted.

As illustrated in FIG. 8, steps S1 is the same as that in the first embodiment illustrated in FIG. 4. In step S2b, the image processing unit 214 acquires phase information of the reference fluoroscopic image 41. In step S3b, the image processing unit 214 acquires phase information of a plurality of fluoroscopic images 40.

In step S4b, from among the plurality of fluoroscopic images 40, the image processing unit 214 does not select the fluoroscopic image 40 having a phase which is different from that of the reference fluoroscopic image as the dissimilar fluoroscopic image 43, and selects the fluoroscopic image 40 having a phase which is the same as that of the reference fluoroscopic image 41 as the similar fluoroscopic image 42. In step S11, the image processing unit 214 creates the device emphasis image 60 by superimposing the selected fluoroscopic image 40 (similar fluoroscopic image 42) having the same phase on the reference fluoroscopic image 41. Steps S12 and S13 are the same as those in the first embodiment illustrated in FIG. 4.

Effects of Third Embodiment

In the third embodiment, the reference fluoroscopic image 41 which is used as a reference is selected from among a plurality of fluoroscopic images 40 which are consecutively generated. The plurality of consecutively generated fluoroscopic images 40 are analyzed, and thus the similar fluoroscopic image 42 similar to the reference fluoroscopic image 41 is selected from among the plurality of fluoroscopic images 40. The image processing unit 214 is configured to create the device emphasis image 60 in which the stent 31 introduced into the subject T is imaged in an emphasized state by superimposing the similar fluoroscopic image 42 on the reference fluoroscopic image 41. Consequently, in the same manner as in the first embodiment, the device emphasis image 60 in which the stent 31 is sufficiently emphasized can be acquired regardless of the disturbance factor.

In the third embodiment, as described above, the image processing unit 214 is configured to select the fluoroscopic image 40 which is acquired in a phase substantially matching a phase of the reference fluoroscopic image 41 as the similar fluoroscopic image 42 from among a plurality of fluoroscopic images 40 on the basis of phase information acquired from the fluoroscopic image 40. Consequently, the image processing unit 214 can easily discriminate the similar fluoroscopic image 42 from the dissimilar fluoroscopic image 43 other than the similar fluoroscopic image 42 without depending on the markers 34a and 34b of the stent 31. As a result, even in a case where the markers 34a and 34b are hardly visually recognized, it is possible to reliably discriminate the similar fluoroscopic image 42 from the dissimilar fluoroscopic image 43 other than the similar fluoroscopic image 42. It is also possible to cope with a phase deviation based on factors other than a heartbeat and breathing unlike a case where matching or mismatching of a phase is determined on the basis of an electrocardiographic waveform acquired from a cardiograph. Consequently, it is possible to more reliably discriminate the similar fluoroscopic image 42 from the dissimilar fluoroscopic image 43 other than the similar fluoroscopic image 42.

### Modification Examples

The disclosed embodiments are only examples and are not intended to be limited. The scope of the present invention is shown not by the description of the embodiments but by the claims, and includes all changes (modification examples) within the meaning and the scope equivalent to the claims.

For example, in the first to third embodiments, as an example, the image processing apparatus 10 (110, 210) used for coronary artery (cardiovascular) intervention treatment has been described, but the present invention is not limited thereto. The present invention may be applied to a radiation image processing apparatus used for applications other than the coronary artery (cardiovascular) intervention treatment. The present invention in which a similar fluoroscopic image can be superimposed on a reference fluoroscopic image is useful in a case of handling a fluoroscopic image of a part where a blood vessel portion is moved among images of the heart periphery.

In the first to third embodiments, a description has been made of an example in which the stent 31 is used as the medical device 30 emphasized in a device emphasis image, but the present invention is not limited thereto. In the present invention, a treatment mechanism introduced into a blood vessel may be used as a medical device instead of a stent. For example, a guide wire or a catheter may be emphasized in a device emphasis image.

In the first to third embodiments, a description has been made of an example in which the present invention is applied to an image processing apparatus which performs image processing on an X-ray image using an X-ray as an example of radiation image processing, but the present invention is not limited thereto. The present invention may be applied to an image processing apparatus for a radiation image using radiation other than an X-ray.

In the present invention, the processes in the first to third embodiment may be combined with each other. For example, the resemblance between a reference fluoroscopic image and a fluoroscopic image may be determined on the basis of the similarity of the fluoroscopic image for the reference fluoroscopic image in a partial image including feature points and the vicinity thereof, described in the first embodiment, and phase information of the fluoroscopic image described in the third embodiment.

In the first to third embodiments, a description has been made of an example in which the device emphasis image 60 is displayed on the display section 7 along with the latest fluoroscopic image 40 (reference fluoroscopic image 41), but the present invention is not limited thereto. For example, only the device emphasis image may be displayed on the display section.

In the first to third embodiments, for convenience of description, a process in the image processing unit has been described by using a flow driven type flow in which processes are sequentially performed according to a process flow, but the present invention is not limited thereto. In the present invention, a process in the image processing unit may be performed according to an event driven type in which a process is performed in the event unit. In this case, a process may be performed according to only the event driven type, and may be performed according to a combination of the event driven type and the flow driven type.

What is claimed is:

1. A radiation image processing apparatus comprising:
an image generation unit that consecutively generates fluoroscopic images based on a detection signal for radiation which is transmitted through a subject; and
an image processing unit that performs image processing on the fluoroscopic images which are consecutively generated by the image generation unit,
wherein the image processing unit is configured to
select a reference fluoroscopic image used as a reference from among the plurality of consecutively generated fluoroscopic images,
analyze the plurality of consecutively generated fluoroscopic images so as to select a similar fluoroscopic image similar to the reference fluoroscopic image from among the plurality of fluoroscopic images, and
superimpose the similar fluoroscopic image on the reference fluoroscopic image so as to create a device emphasis image in which a medical device introduced into the subject is imaged in an emphasized state.

2. The radiation image processing apparatus according to claim 1,
wherein the image processing unit is configured to select the similar fluoroscopic image similar to the reference fluoroscopic image by analyzing the plurality of fluoroscopic images on the basis of feature points of the medical device.

3. The radiation image processing apparatus according to claim 2,
wherein the image processing unit is configured to select, as the similar fluoroscopic image, the fluoroscopic image in which the similarity of a partial image including the feature points of the medical device and the vicinity of the feature points in the fluoroscopic image with respect to a reference partial image including the feature points of the medical device and the vicinity of the feature points in the reference fluoroscopic image is equal to or more than a similarity threshold value.

4. The radiation image processing apparatus according to claim 2,
wherein the image processing unit is configured to select, as the similar fluoroscopic image, the fluoroscopic image in which a movement amount of the feature points of the medical device in the fluoroscopic image with respect to the feature points of the medical device in the reference fluoroscopic image is equal to or less than a movement amount threshold value.

5. The radiation image processing apparatus according to claim 1,
wherein the image processing unit is configured to
acquire phase information of the fluoroscopic image from the fluoroscopic image, and
select, as the similar fluoroscopic image, the fluoroscopic image acquired in a phase substantially matching a phase of the reference fluoroscopic image from among the plurality of fluoroscopic images on the basis of the phase information.

6. The radiation image processing apparatus according to claim 1,
wherein the image processing unit is configured to consecutively update the reference fluoroscopic image so as to consecutively create different device emphasis images.

7. The radiation image processing apparatus according to claim 1, further comprising:
a storage section that stores the fluoroscopic images which are consecutively generated by the image generation unit,
wherein the image processing unit is configured to select the similar fluoroscopic image from among the fluoroscopic images which are stored in the storage section and are generated earlier than the reference fluoroscopic image.

8. The radiation image processing apparatus according to claim 1,
wherein the medical device includes a stent for blood vessel treatment, and
wherein the fluoroscopic image is a radiation image of a part which is periodically moved.

9. A radiation image processing method comprising:
consecutively acquiring fluoroscopic images of a subject through radiation fluoroscopic imaging;
selecting a reference fluoroscopic image used as a reference from the plurality of consecutively generated fluoroscopic images;
analyzing the plurality of consecutively generated fluoroscopic images so as to select a similar fluoroscopic image similar to the reference fluoroscopic image from among the plurality of fluoroscopic images; and
superimposing the similar fluoroscopic image on the reference fluoroscopic image so as to create a device emphasis image in which a medical device introduced into the subject is imaged in an emphasized state.
